(19) United States
(12) Patent Application Publication
(10) Pub. No.: US 2006/0205078 A1
(43) Pub. Date: Sep. 14, 2006

(54) Induction of RNA Interference by Aberrant RNA

(75) Inventors: Demian Obregon, St. Petersburg, FL (US); Mussa Ali, St. Petersburg, FL (US)

Correspondence Address: Demian Obregon, 501 37th Ave. North East, St. Petersburg, FL, 33704, US

(73) Assignee: Demian Obregon, St. Petersburg, FL (US)

(21) Appl. No.: 11/306,044

(22) Filed: Dec. 14, 2005

Related U.S. Application Data
(60) Provisional application No. 60/593,162, filed on Dec. 15, 2004.

(51) Int. Cl. C12N 15/74; C12N 15/63; C12N 15/82

(52) U.S. Cl. 435/455; 435/471; 435/468

(57) ABSTRACT

A method is provided for the induction of aberrant RNA interference (abRNAi) by introducing ‘aberrant’ RNAs into the cell wherein a long-stranded antisense or sense RNA is simultaneously introduced with a short 4-12 mer homologous or complementary or random RNA wherein the long-short RNAs induce sequence specific inhibition of the homologous gene within a cell. The invention disclosed herein can be used to suppress gene expression in vitro, ex vivo or in vivo.

### BACKGROUND OF THE INVENTION

1. Field of the Invention

Invention relates to methods and compositions of inducing RNA interference (RNAi) in the cell by use of aberrant RNA (abRNA) in vitro, ex vivo and in vivo.

2. Description of the Related Art

The RNAi process involves enzymatic processing of long double-stranded RNAs (dsRNAs) produced typically by viral infection, transposons or by introduced vectors or constructs. These long dsRNAs are cleaved into 21-23 nucleotide (nt) pair RNA duplexes by an RNase called Dicer. These duplexes are then incorporated into an RNA-induced silencing complex (RISC). The RISC uses the dicer-produced duplexes to specifically identify and degrade RNA (1). Small interfering RNA (siRNA) mimics dicer processed intermediates in the RNAi pathway. Moreover, due to their small size, less than approximately 28 nt pairs, siRNAs can silence genes by degrading mRNAs in mammalian cells without activating double stranded RNA-dependent protein kinase 1-mediated non-specific suppression (2).

The advantages of RNAi and siRNA include: potency, requiring doses in the nanomole range for effective silencing; stability, as dsRNA molecules typically are more stable than dsDNA; efficacy, typically silencing gene products and subsequent protein products of a gene by greater than 80%. However the advantages of aberrant RNAi (abRNAi) over RNAi and siRNA involve efficiency. abRNAi is more efficient because it utilizes nearly half of the nts required for RNAi and siRNA. abRNAi only requires the provision of only one base strand in the presence of random 4-12 mers. Thus production of synthetic, in vitro transcribed, or in vivo transcribed RNA strands, for use to silence genes can be done at nearly double the efficiency as compared with the classical RNAi and siRNA methods. Thus abRNAi is highly amenable to high throughput processes such as screenings of large numbers of gene.

### SUMMARY OF THE INVENTION

Here we disclose methods for the induction of aberrant RNA interference (abRNAi) by introducing ‘aberrant’ RNA into the cell wherein a long single stranded antisense or sense RNA is simultaneously or consecutively introduced with a short 4-12 nt RNA oligonucleotide (4-12 mer) homologous or random RNA into the cell, wherein the long-short RNA provoke the sequence specific inhibition of the homologous gene within the cell. The invention disclosed herein can be used to inhibit gene expression in vitro, ex vivo or in vivo.

### DRAWING FIGURES

FIG. 1 illustrates the basic design of an aberrant RNA.

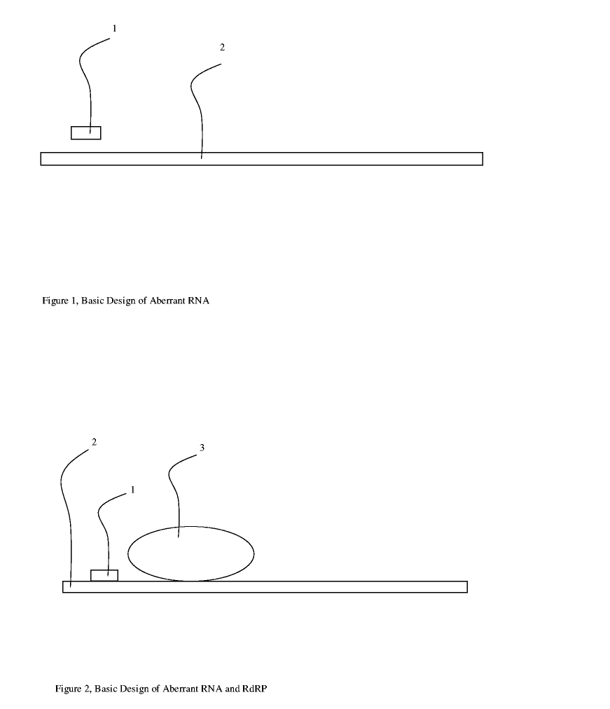

FIG. 2 is a schematic of a putative molecular mechanism involved in abRNAi where an endogenous RdRP acts on the aberrant partial dsRNA duplex to synthesize the dsRNA trigger to provoke RNAi within the cell.

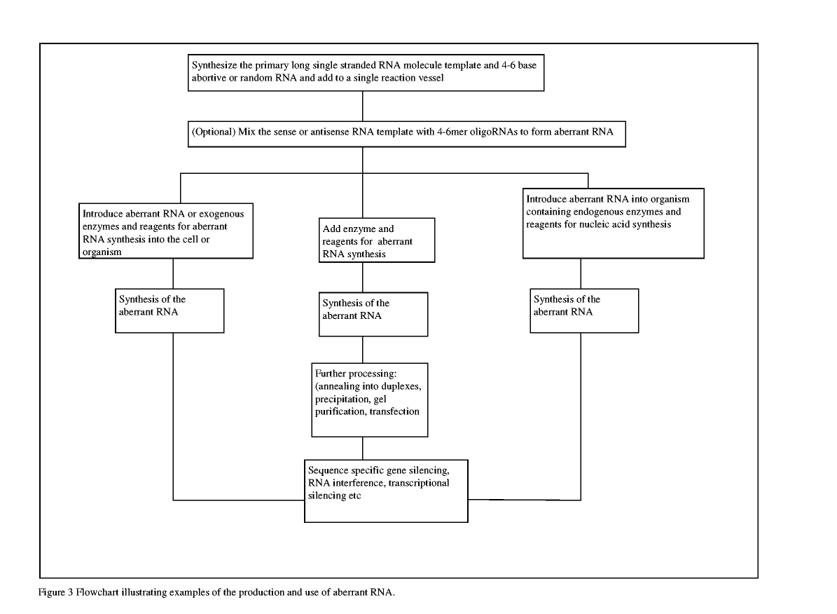

FIG. 3 is a flowchart describing an example of the production and use of aberrant RNA.

FIG. 4 is a graphical representation of the aberrant RNAi model.

FIG. 5 displays representative digital microscopic darkfield images showing GFP expressing Caenorhabditis elegans (C. elegans) treated with or without ‘crude’ sense and RNase A. Also shown are the results from abRNAi silencing experiments.

### REFERENCE NUMERALS IN DRAWINGS

1 is a graphical representation of a species of 4-12 mer random or homologous or complementary short single stranded RNA which are required for the induction of abRNAi which are co-introduced within the cell to effect abRNAi.

2 is a graphical representation of “sense” or “antisense” interfering long single stranded RNA (preferably longer that 20 nucleotides) which is homologous to the gene to be silenced or to its complement which is co-introduced within the cell to effect abRNAi.

3 is a graphical representation of an enzyme with ribonucleic acid dependant ribonucleic acid polymerizing (RdRP) activity.

### DETAILED DESCRIPTION OF THE INVENTION

The present invention provides methods for the induction of aberrant RNA interference (abRNAi) by introducing ‘aberrant’ RNA into the cell wherein a long single-stranded antisense or sense RNA is simultaneously or consecutively introduced with a short single stranded 4-12 mer homologous or complementary or random RNA within the cell, wherein the long-short RNA duplex induces the sequence specific inhibition of the homologous gene within the cell. The invention disclosed herein can be used to inhibit gene expression in vitro, ex vivo or in vivo.

Whereas antisense induced genetic inhibition is often explained by a simple complementarity model where the interfering antisense RNA strand is hypothesized to simply hybridize to the endogenous sense mRNA and interfere with its subsequent translation into protein or leads to its destruction, the oddity of sense RNA induced RNA interference (siRNAi), largely observed in worms and plants, has not been fully explained to date. The possible mechanism involved in siRNAi has been suggested to involve the possibility that a dsRNA intermediate acts as the trigger for the induction of RNAi in cells. However, the precise upstream mechanism(s) of how a single stranded sense RNA homologous to an endogenous gene expressed within the cell enters the dsRNAi pathway or ultimately leads to the sequence specific silencing of the homologous gene is not known.

In worms and plants, sense RNA induced RNAi is induced by the introduction of a transgene expressing an exogenous gene driven under a (viral) promoter or by the introduction of an in vitro transcribed sense transcript directly into cell. In such experiments, while the ‘crude’ in vitro transcribed sense RNA is compatible with the induction of RNAi, a gel-purified sense RNA in vitro transcript is ineffective or substantially less so. The possible mechanism for this disparity in effectiveness has been explained by a model which implicates unintended antisense RNA that is apparently being co-synthesized along with the sense strand during in vitro transcription resulting in a subpopulation dsRNA; the hypothesis further speculates that such dsRNA contaminant in the in vitro preparation, acts as the trigger for RNAi; the putative dsRNA contaminant-trigger in the crude preparation is ‘lost’ upon further gel purification of the sense in vitro RNA transcript, and thus the gel-purified sense RNA looses its capacity to induce RNAi.

While the above model appears plausible, heretofore, a dsRNA contaminant-trigger in a single promoter driven, unidirectional transcription preparation has not been directly detected. In an effort to decipher the ‘crude’ vs. ‘purified’ sense RNA preparation effectiveness in inducing RNAi, we disclose an invention and an easily testable model, which explains the molecular mechanisms involved in the ‘purified’ vs. ‘crude’ sense RNAs induced RNAi. Moreover, as disclosed herein, the present invention could recapitulate abRNAi by the simultaneous introduction of a long-stranded antisense or sense gel-purified RNA with a short 4-12 mer homologous or random RNA wherein the long-short RNAs induce sequence specific inhibition of the homologous gene within the cell.

Bacteriophage RNA polymerases (e.g. T7, T3, SP6) driven in vitro transcripts invariably produce short abortive transcripts (4-12 nucleotide long) which are co-synthesized with the full length RNA transcripts. Recent genetic evidence in both fungi and plants has highlighted the necessary and essential role RNA dependent RNA polymerase (RdRP) plays in siRNAi. In mutant lacking a functional RdRP, siRNAi is defective. Moreover, in cell systems that do not express endogenous RdRP (e.g. Mammalian cells) sense RNA (crude or gel purified) fails to induce RNAi. How the presence of exogenous sense RNA and the endogenous availability of RdRP with in the cell lead to RNAi is not known.

The present invention puts forth a set of experiments and proposes models which delineate the precise mechanism(s) involved in siRNAi and as such discloses how the siRNAi pathway could be artificially utilized to induce abRNAi by recapitulating abRNAi pathway via the introduction of a chemically synthesized short 4-12 mer RNA along with a (gel-purified or chemically synthesized) long sense or antisense single stranded RNA homologous or complementary to the gene to be silenced.

We also disclose herein, that the possible molecular mechanisms involved in siRNAi appear to involve the endogenous activity of RNA dependent RNA polymerase which synthesizes the complementary strand using the interfering long sense or antisense RNA transcript as a template and the abortive short 4-12 mer RNA as primer for de novo synthesis of dsRNA. Such a model predicts that the endogenously and de novo synthesized dsRNA would then enter the RNAi pathway within the cell thereby targeting the homologous gene for degradation.

We also disclose that while abRNA targeting exonic sequences are compatible with silencing, abRNA targeting intronic or promoter sequences are ineffective. We surmise from these observation that abRNAi is a posttranscriptional event largely sequestered within the cytoplasm.

The term “aberrant” RNAs refers to the any RNA that is introduced into the cell wherein the RNA may be in vitro transcribed (before introduction in to the cell) or RNA that is synthesized within the cell via a transgene (preferably driven under abortive transcript generating viral promoter and polymerase, such as T7, flower mosaic virus 35S).

The term “RNA interference” or “RNAi” refers to the art of introduction of dsRNA into the cell by various means and utilizing the RNAi pathway within the cell which ultimately leads to the sequence specific degradation of the homologous mRNA within the cell.

The term “crude” RNA refers any in vitro transcribed RNA or RNA that is synthesized with in the cell by a transgene that is usually driven under a promoter which invariably produced both long transcript and short abortive transcripts.

The term “purified” RNA refers to the long strand RNA transcript gel or further purified to exclude the contaminating abortive transcripts. In some context this term may refer to purified abortive transcripts, as well.

The term “sense RNA induced RNA interference” or “siRNAi” refers to the sequence specific inhibition of a homologous gene with in the cell by the introduction of a homologous sense RNA either directly or via a transgene.

The term “aberrant RNA induced RNA interference” or “abRNAi” refers to the induction of RNA interference within the cell by the introduction of a long (preferably more that 20 nucleotides) sense or antisense RNA with a short (4-12 mer) sense or antisense or random RNA.

A 4-12 mer random or homologous or complementary RNA refers to a species of short RNA which are required for the induction of abRNAi which are co-introduced with in the cell to effect abRNAi.

A “sense” or “antisense” RNA refers to interfering long RNA (preferably longer that 20 nucleotides) which is homologous to the gene to be silenced or to its complement.

The term “homology” or “homologous” refers to sequence identity where the sequences of two RNA or DNA are identical when read 5′-3′ or its complement.

### DESCRIPTION OF PREFERRED EMBODIMENTS

The present invention may be used to introduce aberrant RNA (i.e. a long, single stranded sense or antisense RNA and 4-12 mer random or homologous short single stranded RNA) into a cell for therapeutic purposes. Aberrant RNA targeting a gene associated with either the maintenance of or predisposition to cancer with in the cell or organism could be targeted. In such cases the abRNA may be used to treat cancer that is already in progress or as preventive therapy when the patient is known to have the cancer initiating factor or gene which is inappropriately expressed. The present invention could be used in combination with other treatment modalities, such as chemotherapy, and radiation therapy.

Another embodiment of the present invention may entail silencing a gene required for the maintenance or spread of any pathogen. Such may include genes that the pathogen requires for entry into the host or to replicate within the host. By silencing such essential genes it is envisioned the host could be immunized from future infections or as a therapeutics approach to infection that is in progress.

As disclosed herein, the present invention is not limited to any type of target gene or nucleotide sequence, but may be used to silence any gene in any system where abRNAi are effective or are imbued to be, and where the level of inhibition attained is compatible to the desired outcome.

The present invention could comprise a method for producing plants with reduced susceptibility to insect damage or susceptibility to infection by a pathogen. By targeting the genes which encode factors (host or pathogen) that facilitate the propagation of a pathogen within the plant it is envisioned that such abRNAi mediated intervention would produce a plant more resistant to infection by pathogen.

In another embodiment of the invention, the targeted gene could be an endogenous gene which may be involved in making the plant ripe sooner or have more desirable physical characteristics or make the plant less susceptible to infection. Any such change whether internal or external which renders the plant less prone to infection could be envisioned.

Another preferred embodiment of the present invention may entail an expression construct which may be used to introduce into the plant a transgene which produces the aberrant RNA which has the effect of inhibiting the expression of either an endogenous plant gene or that of the infecting pathogen within the cell. The expression construct could be a conditionally expressed transgene which may be amenable to an outside manipulation. For example, the construct could be expressed under a promoter that requires the availability of an exogenously supplied factor to be expressed. In such embodiment, the abRNA induced silencing within the organism may be tightly controlled.

In yet, another preferred embodiment of the invention it is envisioned that abRNA could be used to target genes that may facilitate crop destruction by insects, nematodes carrying insect pathogens or other organisms. One may target either the infecting pathogen directly or pathogen carrying organisms (such as nematodes) which would result in a plant less susceptible to crop damage.

Another embodiment of this invention may entail the silencing of genes within a host or a microbe dwelling within or on the surface of the host, to promote a symbiotic or beneficial relationship between the host and the microorganism.

The present invention can be used in genetic screening. For example many genes that are known to be encoded within a given genome have unknown function, thus this invention may be used to silence these genes and observe the resulting phenotype, protein expression, signal transduction, or consequential effects on the expression of other genes. Moreover this invention could be used to characterize pharmacological effects, signaling cascades, pathological mechanisms of disease by screening for genes, and thus proteins, necessary for the completion of these processes.

Due to the ease by which abRNA is introduced into a given organism this invention is amenable to high throughput automation whereby abRNAs targeting various genes could be screened for functional effects in large numbers.

The present invention can be used to screen for changes in phenotype, changes in protein expression, changes in non-target gene expression, of genetic polymorphism be targeting sequences specifically including a given polymorphism and then assaying for the resultant change in phenotype, protein expression, or non-target gene expression.

The present invention can also be used to determine the effects of alternatively spiced variants of the same gene by targeting those exons contained within one spice variant but not in another and then observe effects on protein expression, non-target gene expression, signal transduction, biologic function, or other phenotypic characteristics.

The present invention may also be used alone or as a component of a kit. One reagent of this kit would include the abRNA targeting the gene or genes to be silenced, and another reagent of the kit would include a vehicle for the introduction of abRNA into the target organism, or cell type. Also reagents could be provided for the in vitro (without cells) processing of the abRNA. Instructions would be provided to the kit user.

The present invention could be used alone or as a component of a kit. One component of this kit would include enzymes and other reagent necessary to produce abRNA targeting the gene or genes to be silenced, from template RNA or DNA of the user's choice. Another reagent of the kit would include a vehicle for the introduction of abRNA into the target organism, or cell type. Also reagents could be provided for the in vitro (without cells) processing of the abRNA. Instructions would be provided to the kit user.

Another embodiment of this invention may entail its usage as a pesticide where the abRNA would be delivered to a host in order to protect it against a given pest which expresses the target gene, targeted by the administered abRNA. The genes that could be targeted could be those determined to be necessary for survival, proliferation, feeding, growth, or other necessary biological process. Also the pest could be targeted directly via a spray, dust, gas, mist, gel, solid, liquid, or other delivery vehicle.

This invention could be used to engineer an organisms that produce a desired phenotype through inhibition of a target gene or genes are thus allow to produce a product of commercial use. Additionally, this invention could be used to engineer an organism that through the inhibition of the target gene or genes would be better adapted for either commercial use, or survival, or product production.

It is understood the above embodiments are not exhaustive lists of the application and or potential uses of the invention disclosed herein and are merely described as sheer examples. Thus any variations within the scope of this invention would be obvious to those skilled in the art.

The following examples are meant to be illustrative of the present invention; however, the practice of the invention is not limited or restricted in any way by them.

Induction of Aberrant RNA Interference (AbRNAi) in Caenorhabditis elegans

We disclose herein the induction of aberrant RNA interference (abRNAi) by introducing ‘aberrant’ RNAs into the cell: the simultaneous introduction of long single-stranded productive sense RNA with a species of short 2-9 mer homologous and abortive RNAs whereby the long-short RNAs induce sequence specific inhibition of the homologous gene. Additionally, we report the recapitulation of sense RNA induced RNA interference (SI-RNAi) by the artificial co-introduction of a gel-purified long sense RNA with a chemically synthesized 6-nucleotide long short random RNA. A model is proposed in an effort to marry all of the (sense) RNA mediated silencing phenomenon (often carrying varying names) observed in different systems under a single evolutionary conserved ribonucleic acid surveillance pathway and to account for the absolute requirement for the bioavailability of RdRP in the induction of SI-RNAi.

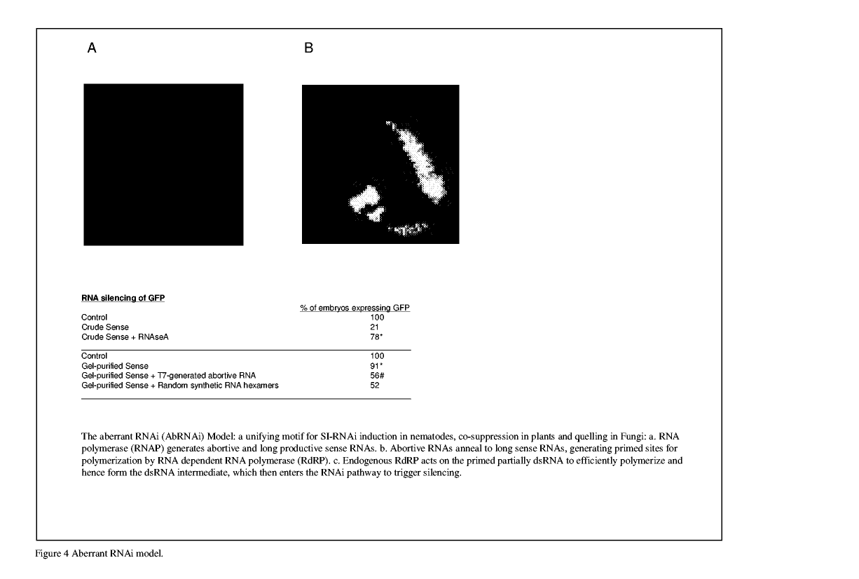

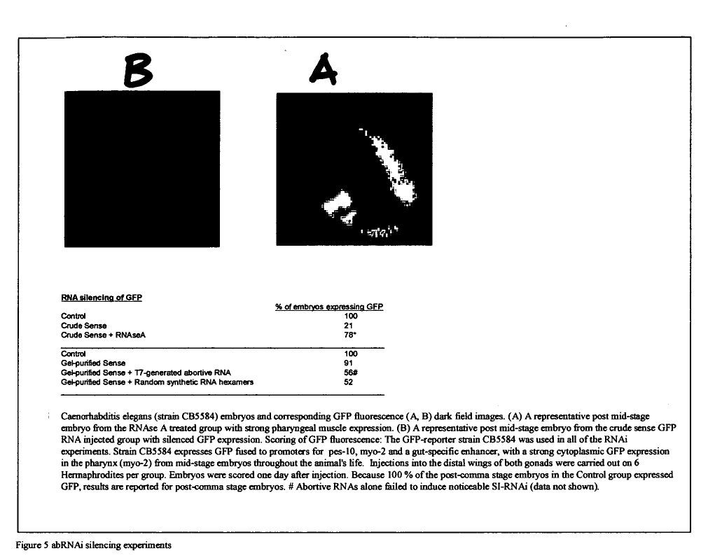

To test a model (FIG. 4) whereby the small species of abortive transcripts act as primers on the long productive sense RNA to drive the efficient de novo synthesis of dsRNA—via endogenous RdRP—that then enters the RNAi pathway: Using Caenorhabditis elegans as a model system, we first directly tested whether our gel-purified sense GFP RNA looses its capacity to induce RNAi. The gel purified sense GFP RNA substantially lost its capacity to induce RNAi (FIG. 5). We then tested whether the abortive transcripts, which are invariably co-generated with the long transcripts, could induce RNAi when co-introduced with the gel purified productive long sense RNA. While the abortive RNAs alone failed to induce noticeable SI-RNAi, the co-introduction of the gel-purified long sense RNA with the small abortive sense transcripts (generated by in vitro transcription using a synthetic oligodeoxyribonucleotide template) is able to induce substantial SI-RNAi (FIG. 5). We then proceeded to recapitulate the role of the abortive transcripts by co-introducing the gel-purified sense GFP RNA with a chemically synthesized random hexamer RNA: the chemically synthesized random hexamer oligoribonucleotide is a potent inducer of SI-RNAi when co-introduced with the long gel-purified GFP RNA (FIG. 5).

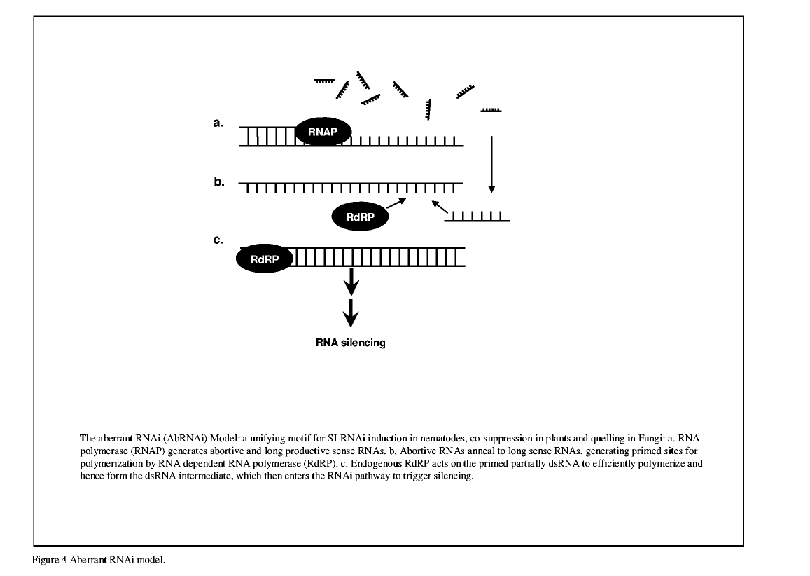

We disclose herein findings that substantiate a model (FIG. 3) delineating the precise upstream molecular mechanism(s) involved in the induction of SI-RNAi, and demonstrate how the RNAi pathway could be artificially engineered to induce abRNAi by recapitulating the abRNAi pathway via the introduction of a chemically synthesized short 2-6 mer ribonucleotides along with a gel-purified (or chemically synthesized) long sense (or antisense) single stranded RNA homologous or complementary to the gene to be silenced. While most of the downstream players of the RNAi pathway have been delineated, a model and experimental evidence to explain the initial induction step in the sense mediated RNAi pathway, heretofore, has been lacking (17-19).

Methods

RNA Synthesis and Gel Purification of Sense RNA:

RNAs were in vitro synthesized with a PCR amplified 520 bp long GFP template. A standard T7 polymerase driven transcription was carried out for 2 hrs. DNA templates were then removed with DNase Treatment. For the RNAse A treated RNAi experiments, the crude RNAs were co-treated with both DNAse and RNAse A. Gel-purification of sense-RNA was carried out by electrophoresis of crude sense RNA preparation on a low-melting agarose gel, followed by phenol-chloroform extraction and isopropanol/ethanol precipitation. Abortive RNAs were synthesized by the procedures of Milligan and colleagues (15). Synthetic random 6 mer RNA were obtained from Dharmacon (Lafayette, Colo.).

Microinjection of RNA:

The microinjections of C. elegans were carried out as described by Mello and colleagues (16). Briefly, after coating a dry agarose injection pad with oil, individual worms are picked off of media plates and transferred to the pad for injection. With a micromanipulator and an inverted compound microscope with DIC optics, the needle loaded with RNA solution (0.5-1 mg/ml) is gently inserted into center of the gonad cytoplasm. Pressure is then applied to the needle, causing the RNA solution to infiltrate the gonad cytoplasm at the point of insertion. After injection, the worms are allowed to recover and are placed on appropriate media plates until further use.

Phenotypic Loss-of-GFP-Expression Analysis:

The GFP-reporter strain CB5584 was used in all of the SI-RNAi experiments. Strain CB5584 expresses GFP fused to promoters for pes-10, myo-2 and a gut-specific enhancer, with a strong cytoplasmic GFP expression in the pharynx (myo-2) from mid-stage embryos throughout the animal's life. Injections of RNAs (0.5-1 mg/ml) into the distal wings of both gonads were carried out on 6 Hermaphrodites per group. Embryos were scored one day after injection. Because 100% of the post-comma stage embryos in the Control group expressed GFP, results are reported for post-comma stage embryos.

### REFERENCES

(1) McCaffrey, A. et al., Nature Biotechnology 21, 639-644 (2003).
(2) McCaffrey, A. et al., Nature 418, 38-39 (2002).
(3) Izant, J. & Weintraub, H. Cell 36, 1007-1015 (1984).
(4) Napoli, C., et al. The plant Cell 2, 279-289 (1990).
(5) Guo, S. & Kemphues, K. Cell 81, 611-620 (1995).
(6) Fire, A., et al. Nature 391, 806-811 (1998).
(7) Fire, A. & Montgomery, M. Trends in Genetics 14, 255-258 (1998).
(8) Jorgensen, R. & Que, Q. Trends in Genetics 15, 11-12 (1999).
(9) Rong, M. et al., Proc. Natl. Acad. Sci. (U.S.A.) 95, 515-519 (1998).
(10) Milligan, J. et al., Nucleic Acid Research 15, 8783-8798 (1987).
(11) Ling, M. et al., Nucleic Acid Research, 17, 1605-1618 (1989).
(12) Forrest, E. et al., Nucleic Acid Research 32, 2123-2128 (2004).
(13) Boutla, A. et al., Nucleic Acids research 30, 1688-1694 (2002).
(14) Baulcombe, D., et al. Science 286, 950-952 (1999).
(15) Milligan, J., et al., Nucleic Acids research 15, 8783-8798 (1987).
(16) Mello, C., et al., Methods Cell Biol 48, 451-82 (1995).
(17) Tomari, Y., et al., Genes and Development, 15, R61-64 (2005).
(18) Jorgensen, R., et al., Trends in Genetics 15, 11-12 (1999).
(19) Gazzani, S. et al., Science 306, 1046-1048 (2004).

What is claimed is:

1. A method to inhibit expression of a target gene in a cell by the introduction of aberrant ribonucleic acid (abRNA), comprising a long sense or antisense single stranded RNA along with a short single stranded RNA (4-12 nucleotides in length), into the cell to inhibit expression of a target gene.

2. The method of claim 1 in which the target gene is a cellular gene, endogenous gene, transgene, viral gene, bacterial gene, protozoan gene, fungal gene.

3. The method of claim 1 in which the cell is from an animal, plant, protozoa, bacteria, or cyanobacteria.

4. The method of claim 3 in which the cell from an animal, plant, protozoa, bacteria, or cyanobacteria is contained within an organism.

5. The method of claim 1 in which the target gene is contained in one organism and the abRNA is expressed by another genetically-engineered or non-genetically engineered organism used to feed the first organism containing the target gene.

6. The method of claim 1 in which abRNA is co-introduced, or co-expressed with enzymes having either RNA replicase activity or RNase activity.

7. The method of claim 6 in which the enzymes having either RNA replicase activity or RNase activity can be cellular, endogenous, exogenous, viral, bacteriophage, bacterial, protozoan, fungal, or genetically engineered.

8. The method of claim 1 in which the abRNA is introduced outside the target cell.

9. The method of claim 8 in the target cell is contained within an organism.

10. The method of claim 9 in which the organism is an animal, plant, protozoa, bacteria, or cyanobacteria.

11. The method of claim 1 further comprising synthesis of the abRNA outside the cell or organism.

12. The method of claim 1 further comprising abRNA synthesis inside the cell or organism.

13. The method of claim 11 in which the cell is from animal, plant, protozoa, bacteria, or cyanobacteria, or the organism is an animal, plant, protozoa, bacteria, or cyanobacteria.

14. The method of claim 12 in which the cell is from animal, plant, protozoa, bacteria, or cyanobacteria, or the organism is an animal, plant, protozoa, bacteria, or cyanobacteria.

15. The method of claim 1 in which the abRNA comprises two separate complementary strands.

16. The method of claim 15 further comprising synthesis of the two complementary strands and initiation of abRNA formation outside the cell or organism.

17. The method of claim 16 further comprising abRNA formation inside the cell or organism.

18. The method of claim 16 in which the cell is from animal, plant, protozoa, bacteria, or cyanobacteria, or the organism is an animal, plant, protozoa, bacteria, or cyanobacteria.

19. The method of claim 17 in which the cell is from animal, plant, protozoa, bacteria, or cyanobacteria, or the organism is an animal, plant, protozoa, bacteria, or cyanobacteria.

20. A method according the claim 1 wherein a kit can be designed for various uses wherein aberrant RNA and other reagents are components.
